(19) United States
(12) Patent Application Publication
(10) Pub. No.: US 2024/0173347 A1
(43) Pub. Date: May 30, 2024

(54) COMPOSITIONS FOR RAISING NAD LEVELS AND METHODS AND USES THEREOF

(71) Applicant: ACCURI, LLC, Augusta, GA (US)

(72) Inventors: Boying DUN, Augusta, GA (US); Jin-Xiong SHE, Augusta, GA (US)

(73) Assignee: ACCURI, LLC, Augusta, GA (US)

(21) Appl. No.: 18/522,970

(22) Filed: Nov. 29, 2023

Related U.S. Application Data
(60) Provisional application No. 63/385,327, filed on Nov. 29, 2022.

(51) Int. Cl. A61K 31/7084; A61K 31/198; A61K 31/455; A61K 31/7004; A61K 31/7012; A61K 31/706; A61P 43/00

(52) U.S. Cl. CPC A61K 31/7084; A61K 31/198; A61K 31/455; A61K 31/7004; A61K 31/7012; A61K 31/706; A61P 43/00

(57) ABSTRACT

Provided herein are compositions and methods of use thereof for efficiently raising NAD levels in a subject in need thereof, and improving the body's functions associated with low NAD levels. The composition comprises NAD precursors and additional components that elevate NAD levels and improve mitochondrial and cellular functions. The additional components include NAD+, NADH, D-ribose and creatine monohydrate, nicotinamide and/or nicotinic acid (NA) and may be added to the formulation to further enhance the composition's efficacy.

### CROSS REFERENCE TO RELATED APPLICATIONS

This application claims benefit of and priority to U.S. Provisional Application No. 63/385,327 filed on Nov. 29, 2022, which is incorporated by reference in its entirety.

### FIELD OF THE INVENTION

This invention relates generally to compositions and methods of use thereof for raising NAD levels in a subject in need thereof.

### BACKGROUND

Nicotinamide adenine dinucleotide (NAD) is a coenzyme found in all living cells. It is essential to life because it catalyzes reactions for more than 500 enzymes, including those involved in the production of cellular energy (ATP). NAD plays an essential role in six hallmarks of aging: DNA repair, epigenetic alteration, loss of proteostasis (proper protein regulation), mitochondrial dysfunction, cellular senescence, and deregulated nutrient sensing.

There are numerous benefits from NAD optimization. The common and important ones include increased energy, better sleep, improved performance, reduction in muscle and joint pains, enhanced immunity against infections, reduced inflammation, reduction in insulin resistance, clearer mental clarity, and improved liver function, just to name a few. Actual benefits may vary from person to person and may depend on the cause of the health conditions.

NAD declines with age and low NAD levels are linked to loss of function and vitality, and many age-related diseases. The vast majority of adults and a small proportion of young children are suboptimal or deficient in NAD, and thus can benefit from NAD optimization. Who needs NAD supplements, and how much someone needs it, can be determined by laboratory tests.

There are potentially many options for raising one's NAD levels. These fall into four different categories. One way to raise intracellular NAD is through supplementation with NAD precursors, the building blocks that cells use to make the NAD molecule. Nicotinamide mononucleotide (NMN) is a one-step precursor to make NAD and nicotinamide riboside (NR) is a two-step NAD precursor. Nicotinamide (NAM) and nicotinic acid (NA) can also be converted into NAD but are much less effective for most people. A few studies in human subjects have evaluated the efficacy of NMN or NR to elevate NAD levels; however, these studies only included very small numbers of subjects. How well these products work are still unknown and could be dependent on the person. Other ways that have been explored to raise NAD levels include the NAD molecule (delivered orally, injections, patches or via IV), inhibition of the enzymes that degrade NAD, and increasing the enzymes that make NAD. The efficacy of these approaches is largely unknown. It is therefore important to develop novel products to elevate NAD levels with high efficiency and in as many people as possible.

Due to the critical roles played by NAD in numerous biological functions as well as the age- and health-dependent decline of NAD levels, optimization of NAD levels is hugely important to maintaining health, performance and longevity. The critical task to formulate a product that can efficiently elevate NAD levels and provide benefits in improving physical, physiological, sexual and/or mental functions.

One commonly used approach to elevate NAD levels has been intravenous (iv) infusion of the NAD molecule. NAD iv infusion has been widely used in clinical medicine for decades and has good efficacy for a number of health conditions and diseases including addictions, neurological diseases such as dementia and infectious diseases including Lyme's disease, COVID-19 and long COVID. However, NAD iv is not able to increase intracellular NAD levels, and therefore not a good approach for increasing cellular functions of NAD.

NAD precursors such as NMN and NR have recently been used as supplements to increase NAD levels. In clinical trials with small number of subjects, both precursors were shown to be able to increase NAD levels. However, these studies did not show the percentage of subjects that experienced significant increases in NAD levels. Data from consumers suggest that NAD levels for many users of these precursors are still not optimized, suggesting that these products may not be optimal.

A few products attempted to improve the efficacy of NMN by adding other supplement ingredients such as Resveratrol and trimethyl glycine (TMG). It is unknown whether the efficacy of these formulations is better than pure NMN.

Therefore, there exists a need for a NAD formulation that can: 1) efficiently elevate NAD levels, and 2) provide benefits in improving biochemical, physical, physiological, sexual, and/or mental health. It is the object of this invention to provide composition that can achieve these goals.

### SUMMARY

It is to be understood that this summary is not an extensive overview of the disclosure. This summary is exemplary and not restrictive and it is intended to neither identify key or critical elements of the disclosure nor delineate the scope thereof. The sole purpose of this summary is to explain and exemplify certain concepts of the disclosure as an introduction to the following complete and extensive detailed description.

In accordance with the purpose(s) of this invention, as embodied and broadly described herein, this invention, in one aspect, relates to a composition for increasing nicotinamide adenine dinucleotide (NAD) levels in a subject in need thereof, comprising an effective amount of a NAD precursor, a NAD compound or the combination thereof. In one embodiment, the NAD precursor is selected from either nicotinamide mononucleotide (NMN) or nicotinamide riboside (NR) or the combination thereof. In another embodiment, the NAD compound is selected from either NAD+ or NADH or the combination thereof.

In one embodiment, the NAD precursors, NAD compounds or the combination thereof are also combined with D-ribose or creatine monohydrate. In one other embodiment, the combination of NAD precursor or NAD compound and D-ribose or creatine monohydrate are further combined with NAM or NA or the combination thereof. In yet another embodiment, the combination of D-ribose or creatine monohydrate with the NAD precursors promotes production of NAD and supports cellular functions.

In another aspect, the invention relates to a method of increasing nicotinamide adenine dinucleotide (NAD) levels in a subject in need thereof comprising administering an effective amount of a NAD precursor, a NAD compound or the combination thereof to optimize NAD and improve health conditions affected by NAD levels. In one embodiment, the NAD precursor is selected from nicotinamide mononucleotide (NMN) or nicotinamide riboside (NR) or the combination thereof. In another embodiment, the NAD compound is selected from NAD+ or NADH or the combination thereof.

In one embodiment, the NAD precursor, NAD compound or the combination thereof is combined with D-ribose or creatine monohydrate. In another embodiment, wherein the combination of NAD precursor or NAD compound and D-ribose or creatine monohydrate is further combined with NAM or NA or the combination thereof.

In another embodiment, the subject has suboptimal or deficient NAD levels. In one other embodiment, the suboptimal or deficient NAD levels are linked to diseases and conditions caused by or associated with, low NAD levels. In another embodiment, the diseases and conditions caused by, or associated with, low NAD levels are selected from a group consisting of addictions, neurological diseases such as dementia, infectious diseases including Lyme disease and COVID-19, long COVID, lipid disorders, (e.g., dyslipidemia, hypercholesterolaemia or hyperlipidemia), stroke, type I and II diabetes, cardiovascular disease, kidney disease, liver disease, chronic fatigue syndrome, lung diseases, autoimmune diseases, obesity, and other physical, physiological, sexual or mental problems.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows longitudinal changes of NAD levels in one 58-year old male user. Baseline NAD levels were determined on and before Jan. 19, 2021 and was found to be very low (less than 24 μM). Supplementation with competitor NAD precursor elevated Intracellular NAD level to 31.9 μM on Feb. 12, 2021, but did not reach the optimum level of 40 μM. Supplementation with 1000 mg of the Vitality Boost, which contains 500 mg of NMN, elevated NAD level to 46.2 μM on Mar. 4, 2021, reaching the optimum (green) zone. Four weeks after stopping supplementation (Apr. 7, 2021), NAD level decreased to 31.9 μM. After increasing the dose to 1500 mg of Vitality powder (750 mg of NMN) for about four weeks, NAD level on May 5, 2021 reached 55 μM. A subsequent wash off period reduced the NAD level to 37.4 μM on Jun. 7, 2021. Dose escalation to 2000 mg of Vitality powder (1000 mg of NMN) increased the NAD level to 62.3 μM on Jun. 14, 2021. Continuous daily supplementation with 2000 mg of Vitality powder kept NAD levels around 60 μM till September 2022.

FIG. 2A shows NAD optimization for a 68-year old female user. Baseline NAD level was determined on Feb. 15, 2021 and found to be 20.9 μM, far below the optimum range. Various NAD precursors from other companies were used with the hope to elevate her NAD level but with no or minimum efficacy. She started daily supplementation with 2000 mg the Vitality Boost (1000 mg of NMN) for four weeks, and her NAD level went up to over 115 μM on Jul. 7, 2021, an increase of over 500%. She then reduced her dosage to 1000 mg of the Vitality Boost powder daily and brought her NAD level down to around 55-70 μM, an ideal level for most people.

FIG. 2B shows reduction of her senescence associated ß-galactosidase as her NAD levels improved.

FIG. 3A shows the NAD levels at baseline (#1 and #2) and two-weeks (#3) and four weeks (#4) post supplementation with the Vitality Boost as well Alanine Aminotransferase (ALT) levels at the same time points for a 43-year old male subject. ALT levels were reduced as NAD levels were increased.

FIG. 3B shows the decline of triglyceride (TRIG) after supplementation in the same subject.

FIG. 4 shows the NAD levels at baseline (#1 and #2) and two-weeks (#3) and four weeks (#4) post supplementation with the Vitality Boost as well Alanine Aminotransferase (ALT) levels at the same time points for a 37-year old male subject. Increase of NAD levels by supplementation with Vitality Boost reduced ALT levels.

FIG. 5 shows the NAD levels at baseline (#1 and #2) and two-weeks (#3) and four weeks (#4) post supplementation with the Vitality Boost as well as Alanine Aminotransferase (ALT) levels at the same time points for a 32-year old female subject. Increase of NAD levels by supplementation with Vitality Boost reduced ALT levels.

FIG. 6 shows a summary of NAD increases in subjects. Baseline NAD levels for all 25 users were measured twice, approximately one week apart. Users took 2000 mg of Vitality Boost for four weeks and their post supplementation NAD levels were measured two- and four-weeks post supplementation. Shown are box plots of NAD levels.

FIG. 7 shows that Vitality Boost outperforms pure NMN by increasing intracellular NAD levels better than NMN alone.

FIG. 8 shows that Vitality Boost (NMN, Creatine, D-Ribose & Nicotinamide) elevated NAD much more than pure NMN.

FIG. 9 shows Vitality Boost optimized NAD for all 5 SubQ NAD+ Users.

### DETAILED DESCRIPTION

The present invention may be understood more readily by reference to the following detailed description of preferred embodiments of the invention and the Examples included therein and to the Figures and their previous and following description.

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the phrase “nutraceutical composition” or variants thereof refers to compositions containing a compound disclosed herein and further containing a food or a liquid, part of a food or a liquid, or is an addition to a food or a liquid, wherein such composition provides medical or health benefits, including the prevention and treatment of disease either alone or in combination with a primary therapy, or the trigger of a beneficial physiological response.

A nutraceutical composition as disclosed herein provides a nutritional source, thus, a nutraceutical composition can be a food product, foodstuff, functional food, or a supplement composition for a food product or a foodstuff. As used herein, the term food product refers to any food which provides a nutritional source and is suitable for oral consumption by humans or animals. The food product may be a prepared and packaged food or an animal feed. As used herein, the term foodstuff refers to a nutritional source for human or animal consumption. Functional foods are foods consumed as part of a diet which are demonstrated to have physiological benefits beyond basic nutritional functions. Food products, foodstuffs, or functional foods include but are not limited to beverages, such as non-alcoholic and alcoholic drinks as well as liquid preparations to be added to drinking water and liquid food, and solid or semi-solid foods. Non-alcoholic drinks include but are not limited to nutritional shakes, soft drinks; sport drinks; fruit juices; and milk and other dairy drinks such as yogurt drinks and protein shakes. Examples of solid or semi-solid food include, but are not limited to, baked goods; puddings; dairy products; confections; snack foods; or frozen confections or novelties; prepared frozen meals; candy; liquid food such as soups; spreads; sauces; salad dressings; prepared meat products; cheese; yogurt and any other fat or oil containing foods; and food ingredients.

As used herein, the term “consists essentially of” (and grammatical variants thereof), as applied to the compositions and methods of the present invention, means that the compositions/methods may contain additional components so long as the additional components do not materially alter the composition/method. The term “materially alter,” as applied to a composition/method of the present invention, refers to an increase or decrease in the effectiveness of the composition/method of at least about 20% or more. For example, a component added to a composition of the present invention would “materially alter” the composition if it increases or decreases the composition's ability to inhibit tumor growth by at least 20%.

As used herein, “pharmaceutically acceptable” means that the material is suitable for administration to a subject and will allow desired treatment to be carried out without giving rise to unduly deleterious adverse effects. The severity of the disease and the necessity of the treatment are generally taken into account when determining whether any particular side effect is unduly deleterious.

As used herein the terms “purified,” or “isolated” refer to a compound after isolated from a synthetic process (e.g., from a reaction mixture), or from a natural source or some combination thereof. Thus, the term “purified,” or its alternatives, including “in purified form” or “in isolated and purified form” refers to the physical state of a compound after being obtained from a purification process or processes as described herein or well known to the skilled artisan (e.g., chromatography, recrystallization and the like), in sufficient purity to be characterizable by standard analytical techniques described herein or well known to the skilled artisan. The purification techniques disclosed herein result in isolated and purified forms of the subject metabolites. Such isolation and purification techniques would be expected to result in product purities of 95 wt % or better, including enantiomers of the same molecule.

As used herein, the terms “prevent,” “preventing,” and “prevention” (and grammatical variants thereof) refer to avoidance, prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods of the present invention. In some embodiments, prevention is complete, resulting in the total absence of the disease, disorder and/or clinical symptom(s). In some embodiments, prevention is partial, resulting in reduced severity and/or delayed onset of the disease, disorder and/or clinical symptom(s).

As used herein, the term “prevention effective amount” (and grammatical variants thereof) refers an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

As used herein, the term “subject” (and grammatical variants thereof) refers to mammals, avians, reptiles, amphibians, or fish. Mammalian subjects may include, but are not limited to, humans, non-human primates (e.g., monkeys, chimpanzees, baboons, etc.), dogs, cats, mice, hamsters, rats, horses, cows, pigs, rabbits, sheep and goats. Avian subjects may include, but are not limited to, chickens, turkeys, ducks, geese, quail and pheasant, and birds kept as pets (e.g., parakeets, parrots, macaws, cockatoos, and the like). In particular embodiments, the subject is from an endangered species. In particular embodiments, the subject is a laboratory animal. Human subjects may include neonates, infants, juveniles, adults, and geriatric subjects. In particular embodiments, the subject is male. In particular embodiments, the subject is female.

As used herein, the term “therapeutically effective” refers to provision of some improvement or benefit to the subject. Alternatively stated, a “therapeutically effective amount” is an amount that will provide some alleviation, mitigation, or decrease in at least one clinical symptom in the subject (e.g., in the case of cancer, reduced tumor size, decreased incidence of metastasis, etc.). Those skilled in the art will appreciate that the therapeutic effects need not be complete or curative, as long as some benefit is provided to the subject.

As used herein, the terms “therapeutically effective amount” and “therapeutically acceptable amount” (and grammatical variants thereof) refer to an amount that will elicit a therapeutically useful response in a subject. The therapeutically useful response may provide some alleviation, mitigation, or decrease in at least one clinical symptom in the subject. The terms also include an amount that will prevent or delay at least one clinical symptom in the subject and/or reduce and/or delay the severity of the onset of a clinical symptom in a subject relative to what would occur in the absence of the methods of the invention. Those skilled in the art will appreciate that the therapeutically useful response need not be complete or curative or prevent permanently, as long as some benefit is provided to the subject.

As used herein, the terms “treatment,” “treat,” and “treating” refer to reversing, alleviating, delaying the onset of, inhibiting the progress of or preventing a disease or disorder. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

As used herein, the term “treatment effective amount” (and grammatical variants thereof) refers to an amount that is sufficient to provide some improvement or benefit to the subject. Alternatively stated, a “treatment effective amount” is an amount that will provide some alleviation, mitigation, decrease, or stabilization in at least one clinical symptom in the subject. Those skilled in the art will appreciate that the therapeutic effects need not be complete or curative, as long as some benefit is provided to the subject.

In one aspect of the present invention, the compositions disclosed herein are “nutraceutical compositions” help control symptoms of disease; alleviate unwanted side effects of an underlying disease or a therapy for such an underlying disease; prevent future disease, or contribute to treatment of a disease. In some embodiments, the nutraceutical composition is administered at the same time as the pharmaceutical treatment; in other embodiments, nutraceutical composition is administered before or after pharmaceutical treatment. When the nutraceutical composition is administered before or after pharmaceutical treatment, such administration occurs hours, days, or months before pharmaceutical treatment. In some embodiments, the nutraceutical composition is administered after one or more symptoms have developed. In other embodiments, the nutraceutical composition is administered in the absence of symptoms. For example, the nutraceutical composition is administered to a susceptible individual prior to the onset of symptoms (e.g., in light of a history of symptoms and/or in light of genetic or other susceptibility factors). Administration of the nutraceutical composition may also be continued after symptoms have resolved, for example, to prevent or delay their recurrence.

### II. Compositions

The compositions provided herein comprise three components consisting of: 1) one or both NAD precursors (NMN and NR) and/or the NAD molecule (NAD+ and/or NADH); 2) D-Ribose and/or creatine; 3) with or without additional ingredients consisting of NAM, NA and/or other ingredients. In one embodiment, disclosed compositions comprise the combination of an NAD precursor and D-ribose, which provides cellular support and can be used to make NMN with endogenous nicotinamide (NAM) or NAM supplied exogenously. The disclosed compositions can also include creatine monohydrate, which is important for muscle mass and function, and other cellular functions. The disclosed compositions can also use the NAD molecule with an NAD precursor (NMN and/or NR) or in place of the NAD precursors together with D-ribose and/or creatine monohydrate. The disclosed compositions can also include NAM and/or niacin, which have important cellular functions and make the NAD production more efficient. The disclosed compositions work synergistically to promote endogenous production of NAD and promote mitochondrial and cellular functions.

### A. NAD Precursors

Compositions comprising an effective amount of an NAD precursor to raise NAD levels in vivo are provided herein. In one embodiment, the NAD precursor can be nicotinamide mononucleotide (NMN) and/or nicotinamide riboside (NR). In a preferred embodiment, the NAD precursor is NMN, a one-step precursor used by cells to make NAD.

In certain embodiments, the compositions provided herein may comprise between about 30% to about 70% of the NAD precursors, NMN or NR (w/w). In some embodiments, the composition may comprise between about 30% to 40%, 30% to 50%, 30% to 60%, or 30% to 70% of NMN or NR. In other embodiments, the composition may comprise between about 40% to 50%, 40% to 60%, or 40% to 70% of NMN or NR. In further embodiments, the composition may comprise between about 50% to 60% or 50% to 70% of NMN or NR. In some embodiments, the composition may comprise between about 60% to about 70% of NMN or NR.

In some embodiments, the compositions provided herein may comprise between about 30% to about 70% of a combination of NAD precursors, NMN and NR. In some embodiments, the composition may comprise between about 30% to 40%, 30% to 50%, 30% to 60%, or 30% to 70% of NMN and NR. In other embodiments, the composition may comprise between about 40% to 50%, 40% to 60%, or 40% to 70% of NMN and NR. In further embodiments, the composition may comprise between about 50% to 60% or 50% to 70% of NMN and NR. In some embodiments, the composition may comprise between about 60% to about 70% of NMN and NR.

i. Nicotinamide Mononucleotide (NMN)

NMN is a one-step and most efficient precursor to NAD. NMN can be converted to NAD by the enzyme nicotinamide mononucleotide adenylyltransferase (NMNAT). In cells, NMN is mainly synthesized from phosphoribosyl pyrophosphate (PRPP) and nicotinamide by the enzyme nicotinamide phosphoribosyltransferase (NAMPT). NMN can also be made from nicotinamide riboside (NR) by the nicotinamide riboside kinase (NRK) enzymes. NMN, NR and NAD can all be degraded, resulting in the generation of nicotinamide (NAM) that can be converted back to NAD via the NAD salvage pathway.

Thus, diseases, disorders and conditions that are affected by increasing NAD levels are likewise affected by the amount of NMN precursor available for NAD biosynthesis, and thus can be treated by administering the NMN compounds and compositions disclosed herein.

NMN works through the nicotinamide mononucleotide adenylyltransferase (NMNAT) pathway or other pathways of NAD+ biosynthesis which have nutritional and/or therapeutic value in improving plasma lipid profiles, prevention of stroke, and/or prolonging life and well-being. Other embodiments relate to a method for preventing or treating a disease or condition associated with the nicotinamide mononucleotide adenylyltransferase (NMNAT) pathway or other pathways of NAD+ biosynthesis by administering a composition comprising NMN. Diseases or conditions which typically have altered levels of NAD or its precursors which can be prevented or treated by supplementing a diet or therapeutic treatment regime with NMN and/or NAD include, but are not limited to, lipid disorders, (e.g., dyslipidemia, hypercholesterolaemia or hyperlipidemia), stroke, type I and II diabetes, cardiovascular disease, and other physical problems associated with obesity.

ii. Nicotinamide Riboside (NR)

Nicotinamide riboside can be used as a precursor to synthesize NMN by the nicotinamide riboside kinase (NRK), and is therefore a two-step precursor to NAD.

NR is found in certain natural products such milk or can be synthesized in laboratories. NR has been used as dietary supplements to increase NAD levels in animals and humans, with improvement in certain physiological and physical functions.

### B. Ribose

Ribose is present as L and D enantiomers. The L-ribose enantiomer is unstable and thus, D-ribose is the primary functional isoform of ribose. D-ribose is highly water-soluble and is present in different types of RNA molecules and the adenosine triphosphate (ATP). D-ribose is also present in the NAD molecule and its metabolites including NMN and NR. Thus, D-ribose is an essential ingredient to make NAD precursors and NAD.

The combination of D-Ribose and Nicotinamide has been used as a supplement to elevate NAD levels. However, the supplement only elevates NAD levels modestly and not in all individuals.

D-ribose by itself may also have important physiological functions and in certain diseases and health conditions such as congestive heart failure (CHF). D-ribose can be supplemented intravenously or orally and has been used in patients and athletes to recharge energy via ATP synthesis.

In certain embodiments, the compositions provided herein may comprise between about 5% to about 40% of D-ribose. In some embodiments, the composition may comprise between about 5% to 10%, 5% to 20%, or 5% to 40% of D-ribose. In other embodiments, the composition may comprise between about 10% to 20% or 10% to 30% of D-ribose. In further embodiments, the composition may comprise between about 20% to 30% of D-ribose.

### C. Creatine Monohydrate

Creatine is one of the natural sources of energy for muscle contraction. About 50% of the body's supply of creatine comes from carnivorous diet including red meat, milk and seafood and the other half is produced in the liver and kidney and then delivered to the skeletal muscle, where 95% of creatine is stored and used for physical activity. Small amounts of creatine are also located in the heart, brain and other tissues.

In certain embodiments, the compositions provided herein may comprise between about 10% to about 50% of creatine. In some embodiments, the composition may comprise between about 10% to 20%, 10% to 30%, 10% to 40% or 10% to 50% of creatine. In other embodiments, the composition may comprise between about 20% to 30%, 20% to 40% or 20% to 50% of creatine. In yet other embodiments, the composition may comprise between about 30% to 40% or 30% to 50% of creatine. In further embodiments, the composition may comprise between about 40% to 50% of creatine.

The most common form of creatine is creatine monohydrate. It is available as a dietary supplement that improves exercise performance, help recovery after intense exercise, prevent or reduce severity of injury, help athletes tolerate heavy training loads. Several studies have shown that users experience sustainably less incidence of cramping, muscle tightness, muscle strains/pulls, non-contact injuries, total injuries and dehydration than those who do not take creatine.

Furthermore, creatine supplements may be helpful to patients with diabetes, heart ischemia, osteoarthritis, fibromyalgia, disorders of creatine metabolism or transport, aging, brain health and neurodegenerative diseases including muscular dystrophy, Parkinson's and Huntington's disease.

### D. Nicotinamide (NAM)

Nicotinamide (NAM), also known as niacinamide, is a form of vitamin B3. It is water-soluble. Nicotinamide, as well as a related molecule called nicotinic acid (NA), is also called niacin. NAM is contained in niacin-rich foods such as fish, poultry, nuts, legumes, eggs, and cereal grains. Nicotinamide has been widely used as supplements.

NAM has also been used to treat niacin deficiencies such as pellagra and skin conditions such as acne and rosacea. Nicotinamide help prevent the occurrence of skin cancer and reduce risk of melanoma.

NAM is an important building block of NAD and its precursors NMN and NR. It has been used in combination with D-ribose to elevate NAD levels in humans with modest outcome.

In certain embodiments, the compositions provided herein may comprise between 0.1% to about 30% of NAM. In some embodiments, the composition may comprise between about 0.1% to 5%, 0.1% to 10%, 0.1% to 15%, 0.1% to 20% or 0.1% to 30% of NAM. In other embodiments, the composition may comprise between about 5% to 10%, 5% to 15% or 5% to 30% of NAM. In yet other embodiments, the composition may comprise between about 10% to 20% or 10% to 30% of NAM. In further embodiments, the composition may comprise between about 15% to 30% of NAM. In certain embodiments, the composition may comprise 0% to about 30% of NAM, indicating that in some embodiments, the composition may not contain NAM.

### E. Nicotinic Acid (NA)

Nicotinic acid (NA), commonly called niacin, is a water-soluble vitamin B3. Although NA and NAM are both called niacin by many people, they are not interchangeable and have very different functions and risk. NA supplements are widely available.

NA is used to prevent and treat niacin deficiency such as pellagra. It is also a supplement and FDA approved drug to lower LDL and raise HDL.

NA can be converted to nicotinamide or NAD and is thus another NAD building block. Both processes take three different steps to convert NA into NAD. NA is usually not a very efficient ingredient in elevating NAD levels and vary significantly from person to person.

In certain embodiments, the compositions provided herein may comprise between 0.1% to about 20% of NA. In some embodiments, the composition may comprise between about 0.1% to 5%, 0.1% to 10%, or 0.1% to 15% of nicotinamide. In other embodiments, the composition may comprise between about 5% to 10%, 5% to 15% or 5% to 20% of NA. In yet other embodiments, the composition may comprise between about 10% to 20% of NA. In certain embodiments, the composition may comprise 0% to about 20% of, indicating that in some embodiment the composition may not contain NA.

### III. Methods of Administration

In some in vivo approaches, the compositions disclosed herein are administered to a subject in a therapeutically effective amount. As used herein, the term “effective amount” or “therapeutically effective amount” means a dosage sufficient to treat, inhibit, or alleviate one or more symptoms of the disease being treated or to otherwise provide a desired pharmacologic and/or physiologic effect. The precise dosage will vary according to a variety of factors such as subject-dependent variables (for example, age, immune system health, diseases, etc.).

In this aspect, the selected dosage depends upon the desired therapeutic effect, on the route of administration, and on the duration of the treatment desired. However, for the disclosed compositions, generally dosage levels of about 2000 mg of the formulation or 1000 mg of NMN. However, the required dosage can be as low as 250 mg of NMN and as high as 2000 mg of NMN daily. The required dosage for NR is similar to those for NMN.

In some embodiments, the disclosed compositions are formulated for enteral administration including oral, sublingual, cream, and rectal delivery, subcutaneous injections, intravenous infusion, preferably orally. In one embodiment, the disclosed compositions are administered in solid dosage form. Suitable solid dosage forms include tablets, capsules, pills, lozenges, cachets, pellets, powders, or granules or incorporation of the material into water, juice, or other beverages.

### A. Formulations for Oral Administration

In some embodiments the compositions are formulated for oral delivery. Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the peptide (or chemically modified forms thereof) and inert ingredients which protect peptide in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. The agents can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push agents out through a single small opening due to osmotic effects.

For oral formulations, the location of release may be the stomach, the small intestine (the duodenum, the jejunum, or the ileum), or the large intestine. In some embodiments, the release will avoid the deleterious effects of the stomach environment, either by protection of the agents (or derivative) or by release of the agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

### EXAMPLES

The disclosed compositions can be used, for example, to elevate NAD levels, improve health, increase energy, increase physical, physiological, sexual and/or mental performance, reduce inflammation, increase insulin sensitivity, improve sleep, boos immunity, reduce likelihood of age-related diseases.

### Example 1: 58 Year-Old Male User

This individual is generally healthy, physically active, had mild form asthma that requires occasional treatment, had joint and back pains due to tennis playing, and had very bad sciatic nerve pain. He started taking daily 1000 mg formulation, escalating quickly to 2000 mg of powder and has stayed on the formulation for 18 months so far.

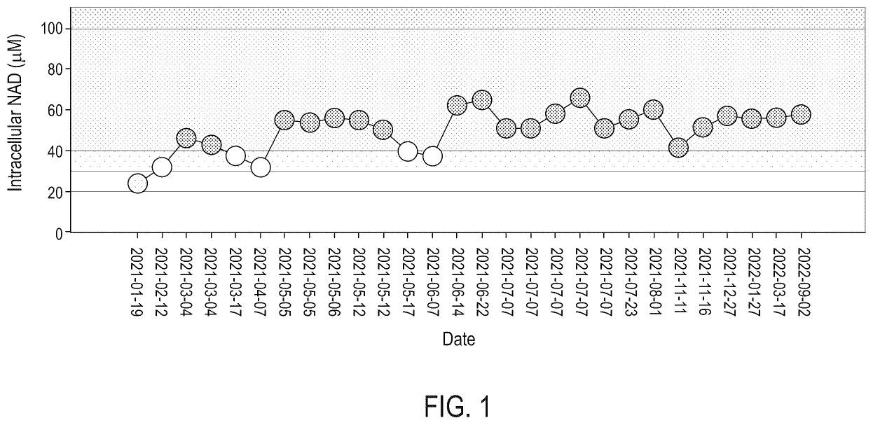

His intracellular NAD level was steadily increased to, and stayed at, around 60 μM (FIG. 1). After approximately three months on the supplement, joint and back pains as well as sciatic nerve pain completely disappeared. His tennis game returned to his level at around 40 years of age. He recovers much more quickly after tennis game and has much greater energy and improved sleep. He did not need any asthma medicine anymore. His biological age as determined by telomere length and methylation is now greatly (7-9 years) younger than his chronological age. His chronic inflammation (hs-CRP), oxidative stress (reactive oxygen metabolites) and cellular senescence burden were all greatly reduced.

### Example 2: 68 Year-Old Female User

This individual is generally healthy and physically active. She started taking 2000 mg of the formulation and reduced dosage to 1000 mg after six weeks.

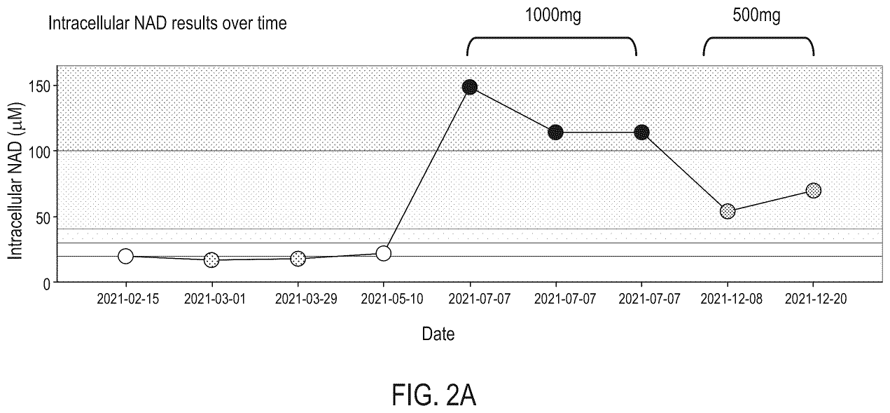

Her intracellular NAD level after four weeks on the supplement was dramatically increased from about 20M to over 115 μM, and came down to 60-75 μM after reducing her dosage to 1000 mg of powder (FIG. 2A). Her energy level and sleep and mental clarity greatly improved and she had new hair growth. Parallel to her increase in icNAD, her senescence-associated ß-galactosidase activity also decreased (FIG. 2B), indicating her senescence burden was reduced, which is a good benefit to her health.

### Example 3: 43 Year-Old Male User

This individual is generally healthy, physically not very active. He started taking the formulation at a dosage of 2000 mg powder for four weeks. He stopped the supplement 3 days before the final laboratory test as he ran out of product.

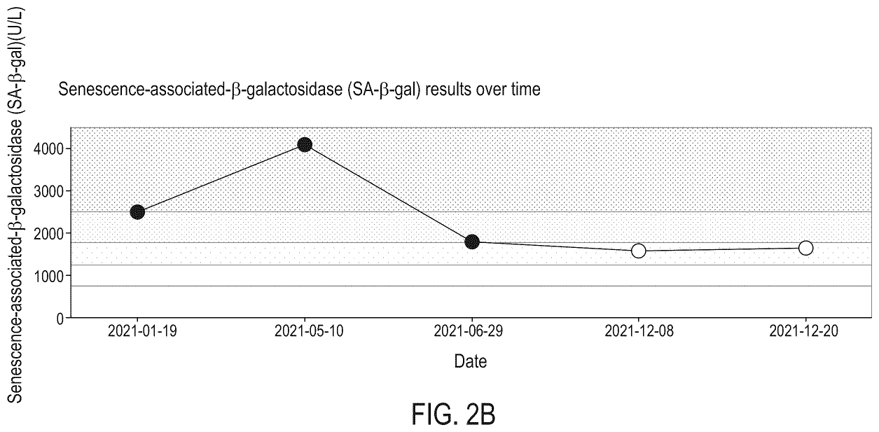

His intracellular NAD level was quickly increased to 84.9 μM two weeks after supplementation and the level came down to 66.5 μM at the four week time point after stopping the supplementation for three days (FIG. 3). He reported more energy and better sleep. His triglyceride level was significantly reduced as well (FIG. 3).

### Example 4: 37 Year-Old Male User

This individual is in good health but had some liver issues. His NAD levels were measured before and after supplementation for two and four weeks.

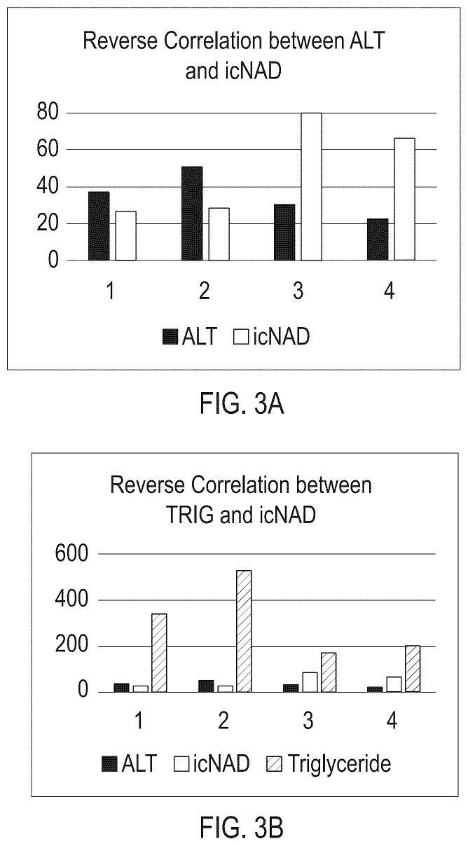

His baseline NAD level was 28.7 μM and, his NAD level increased to 72.1 μM after taking 2000 mg formulation daily for two weeks and further increased to 76.7 μM after four weeks (FIG. 4). In the meantime, his liver enzyme ALT decreased from 63 to 45 two weeks after supplementation and to 42 four weeks after supplementation (FIG. 4). He reported more energy and better sleep with the supplements.

### Example 5: 32 Year-Old Female User

This individual is in good health but have poor sleep. Her NAD levels were measured before and after supplementation for four weeks.

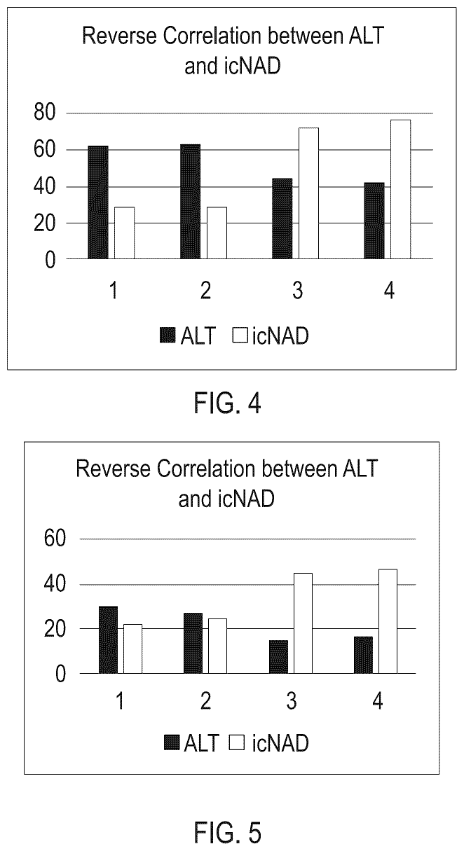

Her baseline NAD levels at two different time points were 21.8 μM and 24.4 μM respectively. Her NAD level increased to 45 μM after daily supplementation with 2000 mg formulation for two weeks, and to 46.7 μM (FIG. 5). Her sleep improved and she had more energy. Her liver enzyme ALT was also decreased as her NAD levels went up (FIG. 4).

### Example 6: Vitality Boost Performance Comparisons

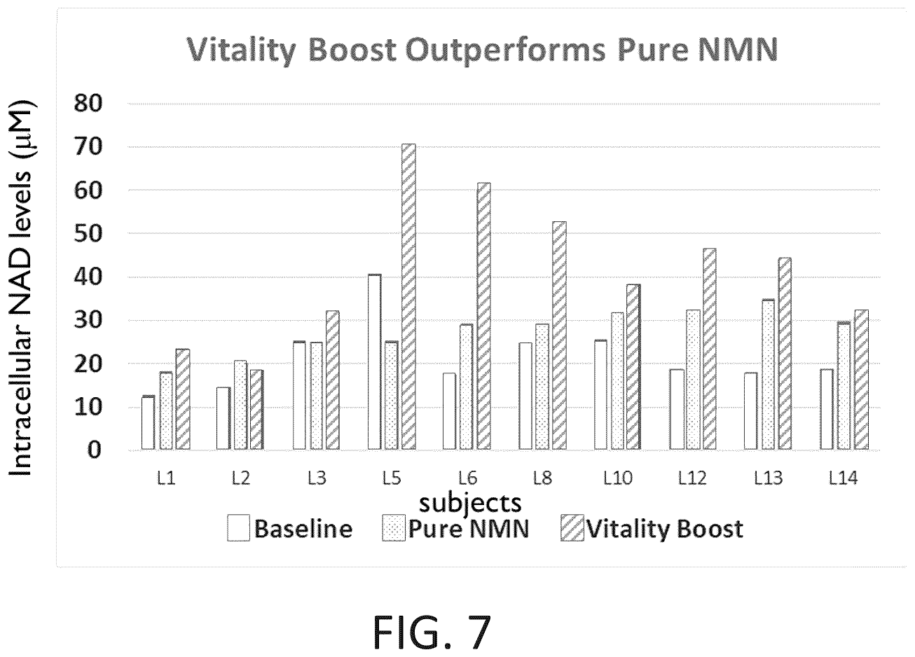

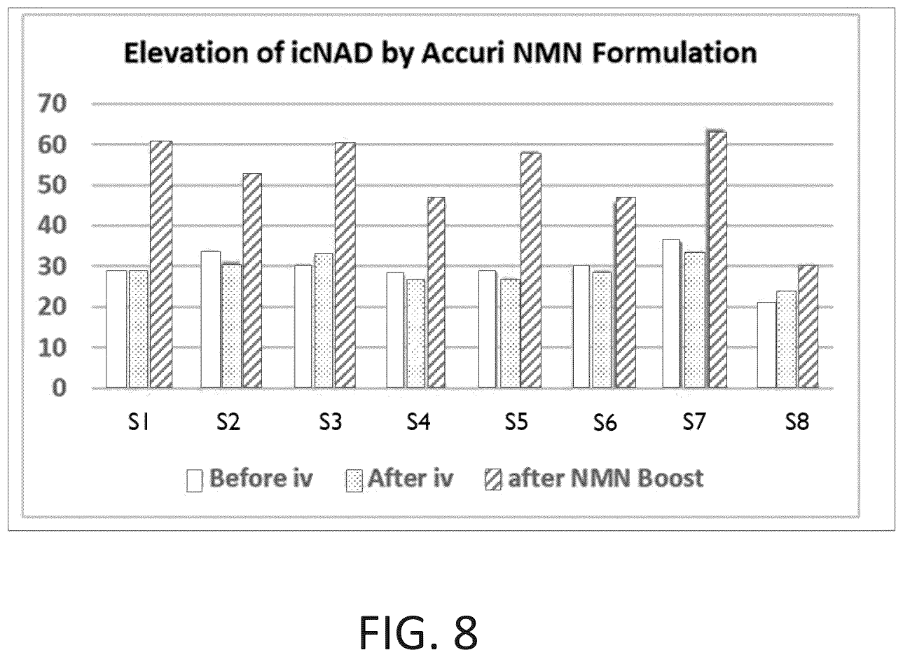

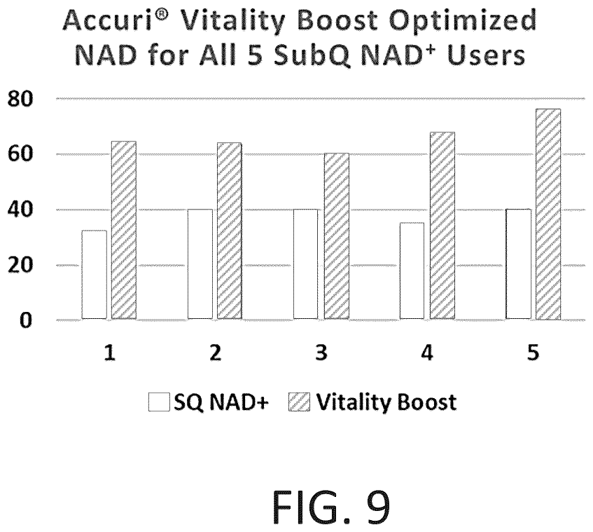

In an experiment comparing the performance of Vitality Boost to pure NMN, intracellular NAD levels were measured at baseline, for 14 subjects who took a pure NMN product, and then switched to Vitality boost for 4 weeks. FIG. 7 shows that Vitality Boost (NMN, Creatine, D-Ribose & Nicotinamide) elevated NAD much more than pure NMN. FIG. 8 shows that intravenous infusion of NAD+(5 sessions of 750 mg) does not increase intracellular NAD levels. In contrast, oral supplementation with 2000 mg of Vitality Boost for four weeks optimized NAD levels in 7 of 8 subjects and significantly boosted NAD in the other individual. FIG. 9 shows Vitality Boost optimized NAD for all 5 SubQ NAD⁺ users who already had slightly higher mean levels than people who never had NAD treatment.

### Example 7: 50 Year-Old Male User

This individual is a profession athlete in good health and is physically very active. He was tired and routinely taking naps in the afternoons. His NAD levels were measured before and after supplementation for four weeks.

His baseline NAD level was 18.4 μM and after taking 2000 mg daily formulation, his NAD level increased to 55.5 μM. He reports much higher energy and does not need naps in the afternoon after his NAD is optimized with the supplement.

### Example 8: Average Increase in NAD Levels

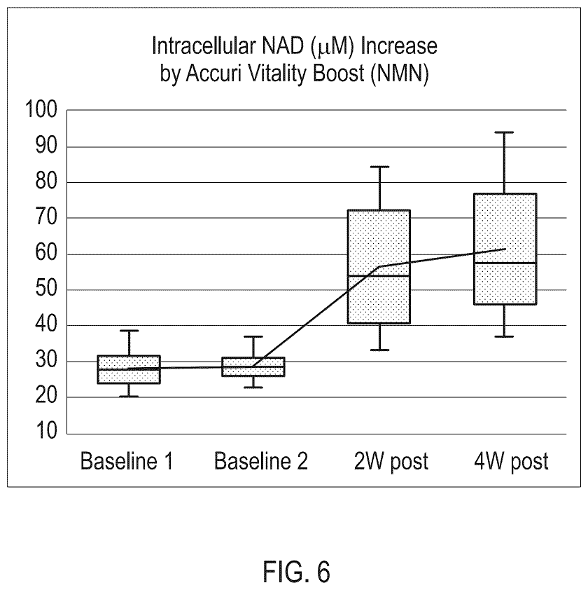

FIG. 6 presents the summary of changes in NAD levels in a group of 25 individuals who had their NAD levels measured at two different baseline time points, two weeks after supplementation and four weeks after supplementation. Every person was supplemented with 2000 mg of the powder for at least four weeks. The mean NAD level was 27.5 μM at the first baseline time point without supplementation and 27.9 μM at the second baseline (FIG. 6). Two weeks after supplementation, the mean NAD level was increased to 55.5 μM, approximately a 100% increase on average. Four weeks after supplementation, the mean NAD level further increased to 59.5 μM.

### Example 9: 39 Year Old Female Using NR Formulation

This 39 year old female individual is in good health. Her NAD level at baseline was 20.9 μM and after taking 2000 mg of the NR formulation consisting of NR, Creatine monohydrate, D-ribose and nicotinamide, her NAD level increased to 51.7 μM. She reports much increased energy, better mental clarity and improved overall performance.

### Example 10: 55 Year Old Female User NR Formulation

This 58 year old female individual is in good health. Her NAD level at baseline was 31.9.4 μM and after taking 2000 mg of the NR formulation, her NAD level increased to 58.5 μM. Her abnormal liver enzyme ALT was reduced from 30 to 18, and triglyceride was also reduced.

### Example 11: 28 Year Old Female User NR Formulation

This 28 year old female individual has a number of health issues. Her NAD level at baseline was 33.5 μM and after taking 2000 mg of the NR formulation, her NAD level increased to 43.6 μM. Her liver enzyme ALT was reduced from a high 73 to 34, a significant improvement.

### Example 12: 56 Year Old Male User NR Formulation

This 56 year old female individual has a number of health issues including type 2 diabetes and hypertension. Her NAD level at baseline was 42.1 μM and after taking 2000 mg of the NR formulation, her NAD level increased to 50.76 μM. Her liver enzyme ALT was reduced from 64 to 53, a significant improvement. Her triglyceride was reduced from 430 to 328.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A composition for increasing nicotinamide adenine dinucleotide (NAD) levels in a subject in need thereof, comprising an effective amount of a NAD precursor, a NAD compound or the combination thereof.

2. The composition of claim 1, wherein the NAD precursor is selected from nicotinamide mononucleotide (NMN) or nicotinamide riboside (NR) or the combination thereof.

3. The composition of claim 1, wherein the NAD precursor is 30-70% of the composition.

4. The composition of claim 1, wherein the NAD precursor is 50% of the composition.

5. The composition of claim 1, wherein the NAD compound is selected from NAD+ or NADH or the combination thereof.

6. The composition of claim 1, further comprising D-ribose or creatine monohydrate.

7. The composition of claim 6, wherein the D-ribose comprises 5-40% of the composition.

8. The composition of claim 6, wherein the creatine monohydrate comprises 10-50% of the composition.

9. The composition of claim 1, further comprising NAM or NA or the combination thereof.

10. The composition of claim 9, wherein the NAM comprises 0.1-30% of the composition.

11. The composition of claim 9, wherein NA comprises 0.1-20% of the composition.

12. The composition of claim 6, wherein the combination of D-ribose or creatine monohydrate with the NAD precursors promotes production of NAD and supports cellular functions.

13. A method of increasing nicotinamide adenine dinucleotide (NAD) levels in a subject in need thereof comprising administering an effective amount of the composition of claim 1 to optimize NAD and improve health conditions affected by NAD levels.

14. The method of claim 13, wherein the subject has suboptimal or deficient NAD levels.

15. The method of claim 14, wherein the suboptimal or deficient NAD levels are linked to diseases and conditions caused by or associated with low NAD levels.

16. The method of claim 15, wherein the diseases and conditions caused by, or associated with, low NAD levels are selected from a group consisting of addictions, neurological diseases such as dementia, infectious diseases including Lyme's disease and COVID-19, long COVID, lipid disorders, (e.g., dyslipidemia, hypercholesterolaemia or hyperlipidemia), stroke, type I and II diabetes, cardiovascular disease, kidney disease, liver disease, chronic fatigue syndrome, lung diseases, autoimmune diseases, obesity, infertility, coma, and other physical, physiological, sexual or mental problems.
